(19) United States
(12) Patent Application Publication
(10) Pub. No.: US 2011/0245919 A1
(43) Pub. Date: Oct. 6, 2011

(54) ADJUSTABLE INTRAOCULAR LENS SYSTEM

(76) Inventor: George H. Pettit, Fort Worth, TX (US)

(21) Appl. No.: 13/075,239

(22) Filed: Mar. 30, 2011

Related U.S. Application Data
(60) Provisional application No. 61/319,292, filed on Mar. 31, 2010.

(51) Int. Cl. A61F 2/16

(52) U.S. Cl. 623/6.22

(57) ABSTRACT

The present invention is related to an adjustable intraocular lens system comprised of a lens body having an adjustable refractive index and a shield for protecting the lens body from degradation that might otherwise be caused by exposure to particular electromagnetic radiation. More preferably, the present invention is directed to an adjustable intraocular lens system comprised of a lens body and a shield wherein the lens body is formed of a material with a refractive index that can be adjusted by exposure to adjusting electromagnetic radiation (e.g., multiple photon energy) and wherein the shield protects the lens body from degradation that might otherwise be caused by exposure to degrading electromagnetic radiation such as ultraviolet radiation.

### CROSS REFERENCE TO RELATED APPLICATION

This application claims priority under 35 U.S.C. §119 to U.S. Provisional Patent Application Ser. No. 61/319,292, filed Mar. 31, 2010, the entire contents of which are incorporated herein by reference.

### TECHNICAL FIELD OF THE INVENTION

The present invention is related to an adjustable intraocular lens system comprised of a lens body having an adjustable refractive index and a shield for protecting the lens body from degradation that might otherwise be caused by exposure to particular electromagnetic radiation. More preferably, the present invention is directed to an adjustable intraocular lens system comprised of a lens body and a shield wherein the lens body is formed of a material with a refractive index that can be adjusted by exposure to adjusting electromagnetic radiation (e.g., multiple photon energy) and wherein the shield protects the lens body from degradation that might otherwise be caused by exposure to degrading electromagnetic radiation such as ultraviolet radiation.

### BACKGROUND OF THE INVENTION

The human eye in its simplest terms functions to provide vision by focusing light onto the retina. This focusing is provided by the cornea (i.e., the clear curved outer portion of the eye) and by the crystalline lens. The quality of the focused image depends on many factors including the size and shape of the eye and the transparency of the cornea and lens.

When age or disease causes the lens to become less transparent, vision deteriorates because of the diminished light which can be transmitted to the retina. This deficiency in the lens of the eye is medically known as a cataract. An accepted treatment for this condition is surgical removal of the crystalline lens and replacement of the crystalline lens by an artificial intraocular lens (IOL).

In the United States, the majority of cataractous lenses are removed by a surgical technique called phacoemulsification. During this procedure, an opening is made in the anterior capsule and a thin phacoemulsification cutting tip is inserted into the diseased lens and vibrated ultrasonically. The vibrating cutting tip liquefies or emulsifies the natural crystalline lens so that the lens may be aspirated out of the eye. The diseased lens, once removed, is replaced by an artificial IOL.

After implantation of the artificial IOL, it is generally desirable for that IOL to provide a high degree of visual clarity to the patient receiving the IOL. The visual clarity provided by the artificial IOL can be dependent upon multiple factors. Particularly important in achieving visual clarity is choosing an IOL with the proper power, which, of course, varies from patient to patient. As such, many systems and devices have been developed for predicting the proper power that an IOL should have for a particular patient. While these systems and devices have been able to make power predictions with a relatively high degree of accuracy, they still often leave the patient with visual clarity that may be less than desired. In addition to power inaccuracies, astigmatism and higher order optical aberrations may also degrade visual clarity.

To address this lack of visual clarity, a variety of measures can be taken. A second surgery can be done to reshape the eye, particularly the cornea to achieve greater clarity. Alternatively, a patient may choose to wear spectacles to address the lack of visual clarity. Both of these options, however, are typically undesirable since patients generally don't want to wear spectacles and don't want to undergo a second relatively invasive surgical procedure.

Recently, a significant amount of research has been expended to develop an IOL with a power that can be adjusted in-vivo (i.e., after implantation). Such power is typically adjusted by adjusting the refractive index of the materials of the

IOL, adjusting the shape of the IOLs, a combination thereof or the like. Examples of IOLs that can be adjusted in-vivo are described in the following references: U.S. Patent Publication No. 2009/0157178 and PCT Publication WO 2005/015268, both of which are incorporated herein, in their entirety, for all purposes.

To impart adjustability to the IOLs, the IOLs must typically include particular materials suitable for adjustment. Such materials, however, can also be particularly susceptible to degradation. For example, some of these materials can significantly degrade upon exposure to ultraviolet radiation.

In view of the above, it would be particularly desirable to provide an adjustable IOL that can be modified to address power and other optic inaccuracies and is less susceptible to degradation.

### SUMMARY OF THE INVENTION

Accordingly, the present invention is directed to an intraocular lens system comprising and intraocular lens body and a shield associated with the intraocular lens body. The intraocular lens body comprises a lens material and an adjustable material distributed within the lens material. The intraocular lens body is sized and shaped to fit into a chamber or capsular bag of an eye of a human being. The intraocular lens has an initial power configured to focus light upon a retina of the eye. Advantageously, the adjustable material, upon exposure to predetermined adjusting electromagnetic radiation, is capable of adjusting the power of the lens by at least one half or even one diopter. The shield is sized and shaped to fit into the chamber or the capsular bag. The shield reflects and/or absorbs predetermined degrading radiation. The adjusting radiation is different than the degrading radiation and typically there is no overlap between the adjusting radiation and the degrading radiation.

In one embodiment, the shield is a layer that is attached to at least a portion of a side of the lens body. The shield can be formed as a layer of material that is dispersed within the lens body in a concentrated manner at one side of the lens body. In a preferred embodiment, the adjustable material breaks or forms bonds upon exposure to the first predetermined electromagnetic radiation for adjusting the refractive index of the adjustable material thereby adjusting the power of the lens. In such an embodiment, the adjusting electromagnetic radiation can be multiple photon radiation. The degrading radiation is often ultraviolet radiation.

The present invention is also directed to a method of surgically implanting an ophthalmic implant within an eye of a mammal. According to the method an incision is created in the eye of the mammal. Thereafter, the intraocular lens system, such as that described above is implanted in the eye of the mammal. Then, if needed or desired adjustments to the power and/or refractive index of the material of the lens body can be made.

### BRIEF DESCRIPTION OF THE DRAWINGS

The accompanying drawings, which are incorporated in and constitute a part of this specification, illustrate aspects of the invention and together with the description, serve to explain the principles of the invention.

FIG. 1 is a side cut away view of an eyeball having an exemplary intraocular lens system in accordance with the present invention.

FIG. 2 is a side sectional view of an exemplary intraocular lens system in accordance with the present invention.

### DETAILED DESCRIPTION OF THE INVENTION

The present invention is predicated upon the provision of an intraocular lens (IOL) system comprised of a lens body and a shield. The lens body is formed of an adjustable material that is capable of changing optical power of the lens body upon exposure to a first predetermined adjusting electromagnetic radiation. The shield is capable of reflecting and/or absorbing a second degrading predetermined electromagnetic radiation. The second predetermined radiation is different from the first predetermined radiation. Further, exposure of the adjustable material of the lens body to the degrading radiation would typically significantly degrade that adjustable material if that material were not protected from such exposure by the shield.

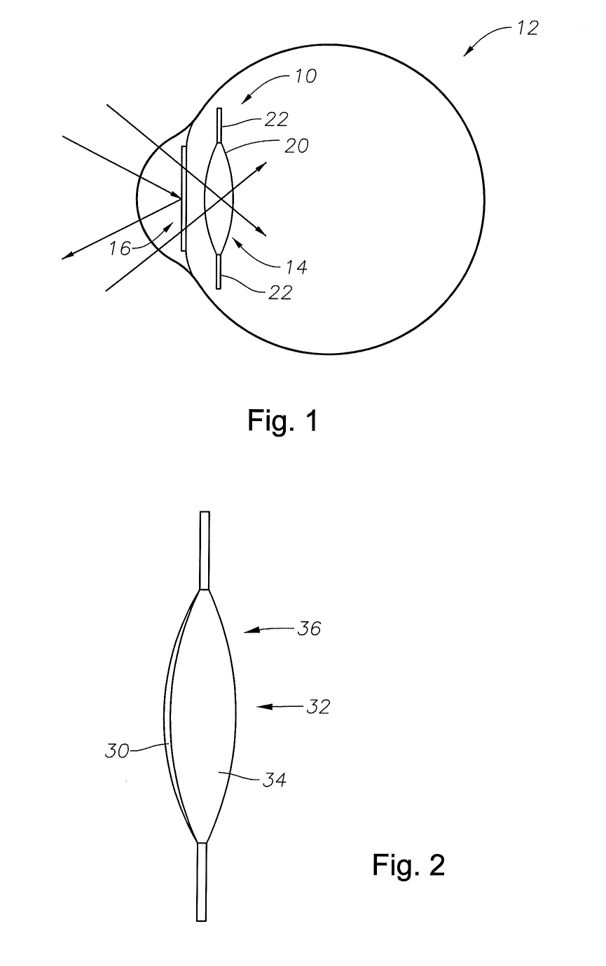

FIG. 1 is an illustration of an exemplary IOL system 10 of the present invention applied to an eye 12 of a mammal, particularly a human being. The system includes an intraocular lens 14 and a shield 16. The lens 14 includes a lens body 20 and haptics 22. The lens 14 illustrated is an aphakic IOL that is designed to replace the natural crystalline lens of the mammal. However, the lens could also be an anterior chamber phakic IOL or a posterior chamber phakic IOL.

The IOL of the present invention, and particularly the lens body of the IOL, is typically formed of a polymer lens material. The polymer lens material is preferably relatively clear and exhibits little or no absorption of light in the visible spectral range under normal conditions (i.e., exposure conditions encountered in everyday life). Of course, the IOL may have some coloration and may be designed to absorb some light (e.g., some blue or violet light) from the visible spectrum. The polymer material is also typically stable at body temperature, i.e. in the range of approximately 30 or 35 to 45° C. Moreover, for ease of processing, the polymer material has a glass transition temperature melting point greater than typical human body temperature (e.g., greater than about 45° C.) such that the material can be processed in liquid or semi-liquid state but also a glass transition temperature and/or melting point that is low enough such that the lens of the material exhibits certain desirable properties (e.g., preferably flexibility). It is also preferable for the lens material to have a relatively high refractive index, thereby allowing for the production of thinner lenses with less material. It is further quite desirable for the lens material to be rollable or foldable such that the lens can be implanted through a relatively small incision in the eye, however, it is contemplated a relatively rigid lens may be encompassed as part of the present invention.

The inventive artificial ocular lens is preferably formed of a polymer material, selected from acrylic polymers, methacrylic polymers, silicone polymers (e.g., silicone elastomers), combinations thereof or the like. In a highly preferred embodiment, the lens is acrylate based. Acrylate based materials are defined as having a substantial portion of acrylate monomers, which are preferably of formulation 1 below:

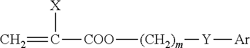

wherein: X is H or CH₃;
m is 0-10;
Y is nothing, O, S, or NR wherein R is H, CH₃, CₙH₂ₙ₊₁(n=1-10), iso-OC₃ H₇, C₆H₅, or CH₂C₆H₅;
Ar is any aromatic ring which can be unsubstituted or substituted with CH₃, C₂H₅, n-C₃H₇, iso-C₃H₇, OCH₃, C₆H₁₁, C₆ H₅, or CH₂C₆H₅;

Suitable monomers of structure (I) include, but are not limited to: 2-ethylphenoxy methacrylate; 2-ethylphenoxy acrylate; 2-ethylthiophenyl methacrylate; 2-ethylthiophenyl acrylate; 2-ethylaminophenyl methacrylate; 2-ethylaminophenyl acrylate; phenyl methacrylate; phenyl acrylate; benzyl methacrylate; benzyl acrylate; 2-phenylethyl methacrylate; 2-phenylethyl acrylate; 3-phenylpropyl methacrylate; 3-phenylpropyl acrylate; 4-phenylbutyl methacrylate; 4-phenylbutyl acrylate; 4-methylphenyl methacrylate; 4-methylphenyl acrylate; 4-methylbenzyl methacrylate; 4-methylbenzyl acrylate; 2-2-methylphenylethyl methacrylate; 2-2-methylphenylethyl acrylate; 2-3-methylphenylethyl methacrylate; 2-3-methylphenylethyl acrylate; 24-methylphenylethyl methacrylate; 2-4-methylphenylethyl acrylate; 2-(4-propylphenyl)ethyl methacrylate; 2-(4-propylphenyl)ethyl acrylate; 2-(4-(1-methylethyl)phenyl)ethyl methacrylate; 2-(4-(1-methylethyl)phenyl)ethyl acrylate; 2-(4-methoxyphenyl)ethyl methacrylate; 2-(4-methoxyphenyl)ethyl acrylate; 2-(4-cyclohexylphenyl)ethyl methacrylate; 2-(4-cyclohexylphenyl)ethyl acrylate; 2-(2-chlorophenyl)ethyl methacrylate; 2-(2-chlorophenyl)ethyl acrylate; 2-(3-chlorophenyl)ethyl methacrylate; 2-(3-chlorophenyl)ethyl acrylate; 2-(4-chlorophenyl)ethyl methacrylate; 2-(4-chlorophenyl)ethyl acrylate; 2-(4-bromophenyl)ethyl methacrylate; 2-(4-bromophenyl)ethyl acrylate; 2-(3-phenylphenyl)ethyl methacrylate; 2-(3-phenylphenyl)ethyl acrylate; 2-(4-phenylphenyl)ethyl methacrylate; 2-(4-phenylphenyl)ethyl acrylate; 2-(4-benzylphenyl)ethyl methacrylate; and 2-(4-benzylphenyl)ethyl acrylate, and the like.

The material of the lens body is typically a polymer formed from at least 10%, more typically at least 30% and even possibly at least 50% acrylate monomers. The material of the body is typically formed from no greater than about 90% acrylate monomers. These acrylate based materials are typically mixed with a curing agent and/or a polymerization initiator so that the materials may be cured to form the IOLs. As such, it will be understood that these monomers are linked to form polymers in the finished IOLs. Examples of acrylate-based lenses are, without limitation, described in U.S. Pat. Nos.: 5,922,821; 6,313,187; 6,353,069; and 6,703,466, all of which are fully incorporated herein by reference for all purposes.

The lens material forming the lens body also typically includes an adjustable material. The adjustable material is typically at least 3%, more typically at least 10% and even more possibly at least 20% by weight of the lens material. The adjustable material is also typically less than 95% and more typically less than 60% by weight of the lens material. The adjustable material, upon exposure to predetermined adjusting electromagnetic radiation, is typically capable of adjusting the power of the lens material and therefore, the power of the lens. Preferably, the material is capable of adjusting the power of the lens by at least 0.5 diopter, more typically at least 1.0 diopter and even possibly at least 1.5 diopter. The adjustable material will change the power of the lens by changing shape of the lens and/or changing the refractive index of the adjustable material.

In one embodiment, it is contemplated that the adjustable material is a polymeric material that undergoes cross-linking upon exposure to the adjusting radiation. Such crosslinking can change the shape of the lens and/or adjust the refractive index of the adjustable material.

In a preferred embodiment, the adjustable material additionally or alternatively undergoes a chemical structure change that results in a refractive index change. In a highly preferred embodiment, the adjustable material of the present invention includes photochemically active groups. When the IOL is exposed to predetermined light at a predetermined wavelength and at sufficiently high photon density (e.g., from multiple (e.g., two) photon light), a photoinduced (e.g., a multiphoton induced) change of the optical properties of the artificial intraocular lens results. A preferred method for this purpose is changing the refractive index of the polymer material by photoinduction. In order to change the refractive index, a number of advantageously two carbon-carbon double bonds are dimerized to form a cyclobutane ring by means of a [2π+2π] cycloaddition under the effect of light. In case a residue of an aromatic π-system is attached to at least one of the C—C double bonds, polarizability in the direction of the double bond strongly decreases due to the fact that resonance with the π-system will no longer be possible upon dimerization. Dimerization or formation, respectively, of the cyclobutane ring thus causes the refractive index to decrease. This effect is even greater if two aromatic π-systems are bonded to the C—C double bond, thereby forming a conjugated system due to the dimerizable double bond. On the other hand, the refractive index can be increased by cleavage of a cyclobutane ring. Examples of such systems are disclosed in U.S. Patent Application 2009/0157178, which is fully incorporated herein by reference for all purposes.

Particularly preferred photochemically active groups are coumarin groups, chalcones, cinnamic acid groups and/or cyclobutane groups.

It is preferable for the photochemically active groups to be covalently bonded to the polymeric material of the intraocular lens, in particular as side chains. It is, however, also possible to provide artificial intraocular lenses made of a polymer material containing molecules with photochemically active groups incorporated or embedded therein.

Artificial intraocular lenses that include polymethacrylic coumarins, polyacrylic coumarins, polymethacrylic cinnamic acid ester, polyacrylic cinnamic acid ester, polyvinyl cinnamic acid ester as well as silicones containing coumarin groups, cinnamic acid groups or/and cyclobutane groups that are covalently bonded thereto are particularly preferred.

One possible lens material is poly(7-methacryloyloxy coumarin) (PMAOC). Poly(7-methacryloyloxy coumarin) may be produced in accordance with known methods (see for example WO 96/10069 or U.S. Pat. No. 2,725,377). In a first reaction stage, 7-hydroxycoumarin is esterified with methacrylic acid chloride to form a reaction product which is then polymerized. Another possible material for the inventive intraocular lenses is poly(vinyl cinnamic acid ester) which may be obtained by a chemical reaction of poly(vinyl alcohol) with cinnamic acid chloride. Still another possible lens material is poly(cinnamoyloxyethyl methacrylate) (PCEM) which is synthesized from hydroxethyl and acrylate which are at first subject to free-radical polymerization to form a reaction product which is then esterified with cinnamic acid chloride. Another possible lens material is formed by mixing and/or reacting one or more of these materials into an acrylate based material.

The inventive lenses and lens materials typically advantageously have a refractive index n of 1.3 to 2.0, more typically of 1.5 to 1.9, and more typically of 1.6 to 1.8 at about body temperature. The change of refractive index that is performable upon the lens or lens material according to the invention is typically at least about 0.001, more typically at least about 0.005 and even possibly at least about 0.01 or 0.017. The change of refractive index that is performable upon the lens or lens material according to the invention is typically less than about 0.1 and more typically less than about 0.05. This change may result in a change in dioptric power that is perfectly sufficient for adjustment in terms of medically relevant cases. If, for example, the refractive index of a lens material of n=1.625 is changed to n′=1.605, this results in a change of the focal length in the aqueous humour (at an assumed refractive index of the aqueous humour of n=1.336, an anterior and a posterior radius of curvature of the lens r1 and r2=20 mm, a thickness of the lens center of 0.8 mm) of f=4.6 cm to f=5.0 cm which corresponds to a change in dioptric power of 21.555 to 20.067. Thus, in this case, a change in dioptric power of approximately 1.5 dpt is obtained.

It is also contemplated that such energy could be employed to adjust the lens for astigmatism. For example, a change in the astigmatic power of the lens body along a preferred or pre-selected axis can be achieved by altering the refractive index about the pre-selected axis in a mirror-symmetric manner or otherwise. It is also contemplated that more complex compensation of higher order aberrations can be achieved as well with appropriate refractive index modification profiles.

In another aspect of the present invention, a change of the focal length of the lens is obtained by structuring a surface or portion of the artificial intraocular lens by photoinduction. In order to do so, only certain areas are provided with photochemically active groups, or only certain areas are exposed to light, thus allowing a photoreaction to occur in these areas only. Advantageously, an effect is obtained that resembles that of a Fresnel lens.

In another additional or alternative aspect of the present invention, a change in shape of the intraocular lens obtained by photoinduction, for example by changing the profile or by elastically deforming the lens in the photoreaction process. This may for example be obtained by photoinduced density changes of the polymeric lens material. Changing the density of the material may for example result in a change in thickness of certain areas of the lens, which consequently leads to a change in curvature.

The intraocular lenses of the present invention may be posterior chamber (P.C.) phakic lenses, anterior chamber (A.C.) phakic lenses or aphakic lenses. Preferably, the IOL is an aphakic IOL configured to replace an individual's natural crystalline lens. The thickness of the lenses usually amounts to 0.8 to 2.0 mm, wherein an optically active area having a diameter of approximately 5 to 7 mm is present within a total diameter of approximately 12 to 13 mm. The lenses typically allow substantially all visible light to pass therethrough although small portions of light from the visible spectrum may be absorbed.

The shield of the present invention typically includes a material, referred to herein as a protective material and more particularly as an ultraviolet protective (UV) material, that is designed to absorb or reflect a very high amount of ultraviolet (UV) light or other degrading electromagnetic radiation. Preferably, the UV material of the shield allows the shield to exhibit very low transmission of UV light. Typically the shield will only allow transmission of less than 10%, more typically less than 1% and even possibly less than 0.1% UV light. Generally, it is preferably that the shield is made entirely or substantially entirely of the UV material. However, other materials may be included as well. As such, it is preferably that the shield is formed of at least 80% and more typically at least 95% by weight of the UV material.

The UV material can be a material that inherently exhibits UV absorption and/or reflection characteristics. Alternatively, the UV material can be a matrix material that includes one or more chromophores. It is also contemplated that the UV material can be a combination of these. For example, the UV material can be a matrix material that exhibits UV absorption and/or reflection characteristics and chromophores can be dispersed within that matrix material.

Examples of chromophores suitable for use in UV material of the present invention include, without limitation, benzophenone-based compounds, benzotriazole-based compounds, cyanoacrylate-based compounds, benzoate compounds and the like. These compounds can be introduced in a matrix material, which is preferably a polymer matrix material. Example of such polymer matrix materials are any of the acrylate, silicone materials discussed herein. The chromophores are typically dispersed throughout a portion or the entirety of the matrix material. Moreover, these chromophore compounds can be reacted into the matrix material or merely trapped within the matrix material. Particularly preferred chromophores are disclosed in U.S. Pat. No. 4,716,234 and U.S. Patent Application Publication No. 20080090937 and U.S. patent application Ser. Nos.: 12/611,539 filed Nov. 3, 2009; 61/223,275 filed Jul. 6, 2009; 61/223,251 filed Jul. 6 2009; and 61/295,900 filed Jan. 18, 2010, all of which are incorporated herein in their entirety for all purposes. When used, the chromophores are typically at least about 3%, more typically at least about 7% and even possibly at least about 10 or even 20% by weight of the UV material. The chromophores are also typically no greater than about 80%, still more typically no greater than about 60% by weight of the UV material.

Examples of materials that exhibit inherent UV absorption and/or reflection characteristics and that are suitable for use as part or the whole of the UV material include, without limitation, polymers such a polyimides and polystyrenes, which may be modified or unmodified.

The shield of the present invention is associated with the body of the IOL. At a minimum, this means that the shield is sized, shaped and otherwise configured to be located in the eye adjacent to the lens body of the IOL. Moreover, the shield will be sized, shaped and configured to be located anteriorly with respect to the lens body meaning that the shield will be located closer to the cornea than the lens body. With reference to FIG. 1, the shield 16 is located in the anterior chamber of the eye 12 while the lens 14 and lens body 20 are located within the capsular bag (not shown) of the eye 12.

In alternative embodiments, and with reference to FIG. 2, a shield 30 of the present invention can be attached to the lens 32, particularly the lens body 34 for forming an intraocular lens system 36 in accordance with the present invention. Such attachment can be accomplished using a variety of techniques. For example, the shield can be overmolded as a layer onto the surface of the lens body or otherwise formed as a layer where there is an intermixing of the material of the shield and the lens body at an interface therebetween. As another example, the lens material and the material of the shield can be concurrently molded in a manner that locates the majority of the shield material, typically 90% by weight of the shield material or more, as a layer on the anterior side of the lens body. As another alternative, the shield may be a separate film that is attached to the lens body as a layer by virtue of an attachment mechanism such as an adhesive, melt sealing, natural or inherent attraction between the shield and lens body or otherwise.

It is possible to change the optical properties of the IOL and particularly the lens body at any time. However, this invention advantageously makes it possible to execute such changes after the lens has been implanted (i.e., in vivo), particularly using the multiple photon (e.g., two photon) photoinduced changes. By delivering the modulating light energy to the eye as a beam tightly focused at the desired location within the IOL, the anterior shield layer, which receives a photon density well below threshold for any 2-photon absorption, is substantially unaffected by and has substantially no impact on the modulating beam. This enables visual acuity to be subsequently adjusted upon implantation or as soon as the eye has recovered from an operation. Moreover, the shield protects the adjustable material from UV radiation that would otherwise cause the lens material, particularly the adjustable material, to undesirably deteriorate and/or degrade (e.g., turn yellow and even possibly brown) and/or to undergo spontaneous refractive index change.

In a preferred embodiment, an intraocular lens body is provided that already consists of a polymer material prior to implantation. Thus, the photoinduced changes can occur without any in-situ (e.g., in vivo) polymerization in the eye or implantation of a monomer material which is to be polymerized in the eye. The lens itself is in fact already formed in advance, and it is only the optical properties of the lens that are changed by photoinduction due to a photoreaction with photochemically active groups. Of course, the lens material may be configured to additionally experience in vivo polymerization. However, a substantial amount (e.g., at least 50%, more typically at least 80%) of the change in optical properties (e.g., power and/or refractive index) will occur due to photo induction of the photochemically active groups.

The photoinduced change of the optical properties preferably occurs by exposure to light covering specific spectral ranges. Since the shield will typically block UV radiation, UV radiation is typically not used. Whatever light is employed, the irradiated light energy is adjusted in a way as to induce a photoreaction of the photochemically active groups, in particular a formation or cleavage of cyclobutane as described above, whilst avoiding an ablation of the lens material. The irradiated energy can however also be adjusted in dependence on the amount of photochemically active groups the material is loaded with, the load preferably amounting to ≧50%, ≧70%, ≧90%, and more preferably to ≧95% of the theoretical value in a covalent bonding situation.

It is highly preferred for the photoinduction to be caused by multiple photon (e.g., two-photon). In this case, a wavelength is irradiated that is in the range of 400 to 1500 nm. In two-photon absorption, an energy density is used that is advantageously in the range of ≧2 kJ cm⁻², more preferably ≧4 kJ cm⁻², and even more preferably ≧5 kJ cm⁻² and up to 20 kJ cm⁻², more preferably up to 10 kJ cm⁻². Radiation is preferably pulsed by means of a laser, the energy density per pulse preferably amounting to ≧50 mJ cm⁻², more preferably ≧100 mJ cm⁻² and up to 300 mJ cm⁻², more preferably up to 200 mJ cm⁻². Likewise, energy is selected in a way as to induce the photochemical reaction whilst avoiding an ablation of the lens material.

In multiple photon (e.g., two-photon) excitation, the wavelength is selected in a way that a single photon does not suffice to induce photochemical activation; in order to obtain the required level of energy, a second photon or more photons must be added to the molecule upon excitation. The photochemical reaction is advantageously induced by two or more photons of the same wavelength. Embodiments comprising two or more photons of different wavelengths may however also prove advantageous in many cases, said embodiments however requiring an increased amount of technical effort. Thus, a specific photon density must be provided for a multiple-photon absorption. Due to the fact that intraocular lenses are worn in the eye and are therefore exposed to light at all times, it is of course essential for the photochemical reaction not to be induced to any significant extent by daylight or sunlight but only if there is a higher photon density. Multiple-photon absorption by means of visible light is a simple way of transporting light through the cornea to the lens, wherein the photon density required to induce the photochemical activation must be higher than that provided by daylight or sunlight. The absorption of multiple photons results in a photochemical activation similar to that provided by short (e.g., UV) wavelength radiation whilst avoiding an unwanted activation by daylight due to the fact that the photon density of daylight is not sufficient for a two-photon excitation. Moreover, the light provided by the multiple photon light source can pass through the UV shield since the wavelength of the light is not absorbed by the UV shield and/or the photon density is insufficient to cause multiple photon absorption at the location of the shield. Thus, the lens body is protected from exposure to UV rays from the sun and other light sources, but still allows for the optical properties of the lens to be adjusted.

As an added advantage, the photoinduced changes can be made to the lenses gradually and/or reversibly. Thus in a first stage, a partial change in refractive index can be obtained by gradual exposure to energy, followed by a subsequent adjustment as soon as the eye has completely recovered. Moreover, it is also possible to fine-tune visual acuity in a gradual manner. Moreover, the refractive index may be selectively increased or reduced, respectively, by systematic cleavage or formation of cyclobutane groups via exposure to the wavelength that is suitable for the particular process, thereby causing a change in the range of +dpt or −dpt, respectively.

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

Other embodiments of the present invention will be apparent to those skilled in the art from consideration of the present specification and practice of the present invention disclosed herein. It is intended that the present specification and examples be considered as exemplary only with a true scope and spirit of the invention being indicated by the following claims and equivalents thereof.

We claim:

1. An intraocular lens system, comprising:
an intraocular lens body comprising a lens material and an adjustable material distributed within the lens material, the intraocular lens body being sized and shaped to fit into a chamber or capsular bag of an eye of a human being, the intraocular lens having an initial power configured to focus light upon a retina of the eye, wherein:
i) the adjustable material, upon exposure to predetermined adjusting electromagnetic radiation, is capable of adjusting the power of the lens by at least one diopter; and
a shield associated with the intraocular lens body, the shield being sized and shaped to fit into the chamber or the capsular bag, wherein:
i) the shield reflects and/or absorbs predetermined degrading radiation; and
ii) the adjusting radiation is different than the degrading radiation.

2. A system as in claim 1 wherein the shield is a layer that is attached to at least a portion of a side of the lens body.

3. A system as in claim 1 wherein the shield is formed as a layer of material that is dispersed within the lens body in a concentrated manner at one side of the lens body.

4. A system as in claim 1 wherein the adjustable material breaks or forms bonds upon exposure to the first predetermined electromagnetic radiation for adjusting the refractive index of the adjustable material thereby adjusting the power of the lens.

5. A system as in claim 1 wherein the adjusting electromagnetic radiation is multiple photon radiation.

6. A system as in claim 1 wherein the degrading radiation is ultraviolet radiation.

7. A system as in claim 1 wherein there is no overlap between the adjusting radiation and the degrading radiation.

8. A system as in claim 1 wherein the shield is formed of a matrix material with chromophores dispersed in the matrix.

9. A system as in claim 8 wherein the chromophores are benzotriazoles.

10. A system as in claim 1 wherein the lens body includes a substantial amount of acrylate.

11. A system as in claim 1 wherein the shield and the lens body are foldable.

12. An intraocular lens system, comprising:
an intraocular lens body comprising a lens material and an adjustable material distributed within the lens material, the intraocular lens body being sized and shaped to fit into a chamber or capsular bag of an eye of a human being, the intraocular lens having an initial power configured to focus light upon a retina of the eye, wherein:
i) the adjustable material, upon exposure to predetermined adjusting electromagnetic radiation, is capable of adjusting the power of the lens by at least one diopter; and
a shield associated with the intraocular lens body, the shield being sized and shaped to fit into the chamber or the capsular bag, wherein:
i) the shield reflects and/or absorbs predetermined degrading radiation; and
ii) the adjusting radiation is different than the degrading radiation;
wherein:
i) the adjustable material breaks or forms bonds upon exposure to the first predetermined electromagnetic radiation for adjusting the refractive index of the adjustable material thereby adjusting the power of the lens;
ii) the adjusting electromagnetic radiation is multiple photon radiation; and
iii) the degrading radiation is ultraviolet radiation; and
iv) there is no overlap between the adjusting radiation and the degrading radiation.

13. A system as in claim 12 wherein the shield is a layer that is attached to at least a portion of a side of the lens body.

14. A system as in claim 12 wherein the shield is formed as a layer of material that is dispersed within the lens body in a concentrated manner at one side of the lens body.

15. A system as in claim 12 wherein the shield is formed of a matrix material with chromophores dispersed in the matrix.

16. A system as in claim 15 wherein the chromophores are benzotriazoles.

17. A method of surgically implanting an ophthalmic implant within an eye of a mammal;
creating an incision in the eye of the mammal;
implanting the intraocular lens system of claim 1 into the eye of the mammal.

18. A method as in claim 17 further comprising adjusting the refractive index of at least a portion of the lens body after the implanting of the system wherein the mammal is a human being.

19. A method of surgically implanting an ophthalmic implant within an eye of a mammal;
creating an incision in the eye of the mammal;
implanting the intraocular lens system of claim 12 into the eye of the mammal.

20. A method as in claim 19 further comprising adjusting the refractive index of at least a portion of the lens body after the implanting of the system wherein the mammal is a human being.
